(19) United States
(12) Patent Application Publication
(10) Pub. No.: US 2004/0115784 A1
(43) Pub. Date: Jun. 17, 2004

(54) Apparatus and method for streaming electroporation

(75) Inventor: Sergey Dzekunov, Germantown, MD (US)

Correspondence Address: FULBRIGHT & JAWORSKI L.L.P., 600 CONGRESS AVE., SUITE 2400, AUSTIN, TX, 78701, US

(73) Assignee: Maxcyte, Inc.

(21) Appl. No.: 10/675,592

(22) Filed: Sep. 30, 2003

Related U.S. Application Data
(60) Provisional application No. 60/414,974, filed on Sep. 30, 2002.

(51) Int. Cl. C12N 13/00; C12M 1/42

(52) U.S. Cl. 435/173.6; 435/285.2

(57) ABSTRACT

Techniques for streaming electroporation. A representative but non-limiting method includes: generating a spatially inhomogeneous electric field with a pair of electrodes and displacing the pair of electrodes and a sample relative to one other while the electric field is substantially constant in terms of magnitude so that the sample is displaced across electric field lines for a time sufficient to effect electroporation.

This application claims priority to, and incorporates by reference, U.S. Provisional Patent Application Serial No. 60/414,974, which was filed on Sep. 30, 2002.

### BACKGROUND

1. Field of the Invention

The present invention relates to methods and apparatus for the electrical treatment of cells or particles and especially for the introduction of biologically active substances into various types of living cells by means of electrical treatment. More particularly, the present invention relates to methods and apparatus for the introduction of biologically active substances into various cells or particles suspended in a fluid by the electrical treatment commonly known as electroporation to achieve therapeutic results or to modify cells being used in research to increase their experimental utility. Electroporation is presently used on cells in suspension or in culture, as well as cells in tissues and organs.

2. Description of Related Art

Electroporation (“EP”) is a technique that is used for introducing material such as biologically active substances into biological cells or cell-like particles, and is currently performed by placing one or more cells, in suspension or in tissue, between two electrodes connected to an electrical power supply that is capable of supplying high-voltage pulses to the electrodes. The high voltage pulses are commonly produced by the timed discharge of one or more capacitors. The strength of the electric field applied to the electrodes and thereby to the suspension and the duration of the pulse (the time that the electric field is applied to the electrodes and thereby also to a cell suspension) is varied by the practitioner according to the type of cell being electroporated to optimize electroporation. Effective electroporation occurs when an optimal set of conditions, which depend on the sample being electroporated, exist. Samples are exposed to a pulse for such a length of time and at such a voltage as to create an electric field that leads to the formation of transient pores in membranes of the sample. The strength or magnitude and the duration of the high voltage pulse applied to the electrodes determines, together with the dimensions and spacing of the electrodes and electrical properties of the sample, the magnitude and duration of the electric field applied to the cell. The magnitude and duration of the pulse applied to the electrodes is chosen to maximize electroporation of the cells. Through the transient pores, material such as biologically active substances can enter the cell by diffusion, by electrophoretic transfer, or both.

As a method of introducing biologically active substances into cells, electroporation offers numerous advantages: it is safe (no chemicals or virus-derived materials need to be used); it can be used to treat whole populations of cells essentially simultaneously; it can be used to introduce essentially any macromolecule, especially DNA, into a cell; and it can be used with a wide variety of primary or established cell lines and is particularly effective with certain cell lines. Applications of electroporation include, by way of example, gene/cell therapy, protein production, target validation, and gene screening.

Practically all of the existing electroporation procedures make use of high voltage (HV) pulses delivered to metal electrodes and from those electrodes to either cell suspensions (in vitro or ex vivo), adherent cells, or to tissues (in vivo). Processing cells in suspension allows superior control over the procedure and is the most preferred method in research applications.

As generally practiced in vitro, electroporation is carried out in small (less than 0.5 milliliters) cuvette-like chambers containing a pair of electrodes with motionless cells and fluid (“static” EP). These static EP methods are not suitable for processing large volumes of sample. The limited volume of chambers for static EP determines the maximal amount of cells that can be conveniently electroporated. Simply increasing the physical dimensions of chambers is not feasible due to the need for even more expensive HV pulsers with greater current and/or voltage capabilities. Static EP devices electroporate enough cells for many laboratory research applications but not nearly enough for either industrial applications or cell-based therapy. The latter often deal with tens of liters of cell culture while the former can make use of hundreds of milliliters of blood cells. Theoretically, large volumes could be electroporated by pooling large numbers of small batches from static electroporation. This, however, would be very time consuming or require simultaneous use of multiple electroporation apparatuses which would be costly and exacerbate problems of reproducibility and quality assurance. Such an approach is not a realistic option for industrial applications or cell therapy. Therefore, a need exists for a higher throughput system capable of processing large volumes of sample over a short period of time.

To address some static electroporation problems, an apparatus was designed to permit cells to be electroporated while they flow between two electrodes (flow EP). An HV pulse is applied to batches of cells that pass between the electrodes (see FIG. 1). Such a technique is more convenient at least because it is especially useful when large volumes of cells must be electroporated.

The application of an electric field (EF) to cells in conventional flow EP is typically the same as for static EP: a pulse of electrical energy is applied at certain time intervals that are long when compared to the time duration of each individual pulse (a quantitative measure of the ratios of times is discussed below). In conventional flow EP, computer-controlled electronic switches typically close repeatedly to deliver distinct HV pulses to a new batch of cells once a prior batch of cells are displaced by a pump out of the space between electrodes. In some respects, therefore, conventional flow EP processes are similar to static EP—in the way that EF is applied to the electrodes and to the sample. The two processes differ, however, in the way samples are handled one is static while the other is characterized by batch-wise flowing.

In both static and conventional flow EP methods, the transient nature of the electric field experienced by the sample being electroporated is the result of electronic control over the magnitude and duration of one or more voltage pulses applied to the electrodes. In the case of flow EP, the flow rate of cells between the electrodes must be coordinated with the rate of high-voltage pulse application.

Although flow EP shows several advantages over static EP, room for improvement remains.

First, controlling the strength and duration of a transient electric field (i.e. electric pulses) often requires complex electronic circuitry that takes a long time to charge. Electrical power units capable of producing controlled pulses can become exceptionally costly and bulky if they must operate at an increased rate in a high-throughput system. Energy for pulsing is generally provided by discharging a bank of capacitors. The amount of energy available in those banks must be proportional to the volume of cells being electroporated with each discharge. Consequently, the larger the volume, the longer it takes to accumulate sufficient energy. Accordingly, it would be advantageous to provide for methods that require less complex circuitry and which do not exhibit such a dependence on recharge times.

Second, although throughput is greater in conventional flow EP chambers, it is still limited. Throughput refers generally to the amount of sample (e.g., cell suspension) processed in certain amount of time. Since it takes a certain amount of energy to electroporate a unit of volume of cell suspension, the more volumetric units that are processed in a given time, the more energy in the same time is consumed. Therefore, the speed, or the throughput, of a process can be defined (and limited) as the rate of energy consumption, or power, which is defined as the ratio of energy to time. As throughput is increased, the electronics may not be able to cope with the requirements of either the instantaneous power consumption (if the volume being pulsed at once is too large) or the average power consumption (if the pulsing also must be done at very short intervals). Accordingly, it would be advantageous to provide for methods that increase throughput while not burdening electronic subsystems.

Third, conventional flow EP can generate excessive heat. It can be shown that heat is normally produced as a side effect of practically every electroporation process, and that heat may cause irreversible damage to biologic material or live cells. Electroporation can cause heating of cells by 10-20 degrees Celsius above room temperature. To minimize this increase in temperature, and to minimize its duration, cooling is sometimes applied to suspensions of cells being electroporated, especially in conventional flow EP processes. It is known that heat is transferred primarily by diffusion and this limits the rate of cooling. This places another set of practical limitations on the scale at which ordinary flow electroporation of cells may be carried out. Accordingly, it would be advantageous to provide for methods that generate less heat or deal with heat more effectively.

Fourth, conventional flow EP suffers from very low duty cycles, which can represent, among other things, a significant amount of “down time” during a process. In any conventional EP application the combined duration of HV pulses that each cell must experience in order to achieve the desired effect is very short in the time-scale of the entire laboratory procedure (normally in the range of 10⁻⁵-10⁻² seconds). Replacement of a batch of cell suspension between subsequent pulses or pulse bursts typically takes several seconds. As illustrated on FIG. 2, a first batch of cells has received its very short pulse, and the second pulse will be applied only when the second batch of cells replaces the first one, i.e. in several seconds or more. Here a single pulse has been considered for simplicity; of course a pulse train can be used without altering the basic principle.

Comparison of these characteristic times (milliseconds for the duration of a pulse or the combined duration of several pulses compared to several (e.g., ten) seconds to replace the contents of a channel) shows that in a typical flow EP application, the electroporation itself (actual time spent pulsing) occupies a very small fraction of any overall procedure time. This fact can be illustrated by a numerical ratio of the time during which electric field strength is sufficient to cause electroporation to the time a sample is between electrodes (procedure time), or an equivalent technical term called “duty cycle.” It can take any value from zero to 100 percent and correspondingly can refer to either very short pulses/long intervals or long pulses/very short intervals. The electronic subsystem of a conventional flow EP system is idle for a relatively long time during the volume replacement; therefore as in static EP, the duty cycle of current flow EP is extremely small. The duty cycle also indicates how often an electrode or electrodes are energized. The lower the duty cycle, the longer the delay between energized states. In view of the above, it would be advantageous to provide for methods that provide higher duty cycles to, among other things, make the EP process more efficient.

Referenced shortcomings of conventional methodologies mentioned above are not intended to be exhaustive, but rather are among many that tend to impair the effectiveness of previously known techniques concerning electroporation. Other noteworthy problems may also exist; however, those mentioned here are sufficient to demonstrate that a need exists for the techniques described and claimed here.

### SUMMARY OF THE INVENTION

Shortcomings of conventional methodologies are reduced or eliminated by the techniques disclosed here. These techniques are applicable to a vast number of applications, including but not limited to applications involving flow-based electroporation.

Procedures described here are able to decrease complexity of necessary electronic circuitry, increase throughput, and increase duty cycles of flow-based electroporation devices. Moreover, the techniques described here provide for methods and associated apparatuses that allow electroporation to be carried out faster, at larger scale, and at lower cost than presently possible.

Embodiments of the present invention involve a new basic principle of controlled exposure of biological material to electrical field, and electroporation in particular. Control of the magnitude, and particularly the duration of the electric field that is applied to a sample is generally determined not by changing the magnitude of the electric field applied to a pair of electrodes, but rather by having the sample pass between a pair of electrodes, the duration of the period during which the sample is substantially between the electrodes determining the duration of the electric field applied to the sample. During the passage of particular particle between the electrodes, the magnitude of voltage is substantially constant.

According to this principle, the duration of exposure of each biological cell to EF can be controlled by the cell's movement through the electrical field instead of switching the voltage ON and OFF in a power supply.

A major significance of this approach is that it provides a simultaneous and reciprocal increase in the process duty cycle and the decrease in instantaneous power consumption, making the entire EP application of a low-power type and rendering the same or higher overall throughput.

Thus, embodiments of the present invention overcome drawbacks inherent to existing electroporation methods by providing a simpler, faster and less expensive method for introducing biologically-active substances and genetic material into cells, which can be scaled up to almost any desired volume of biological material while maintaining sterile conditions.

From the point of view of apparatus fabrication, it may be most convenient to flow cells between stationary electrodes (the electrodes being stationary relative to the apparatus as a whole); however, the method may be carried out using an apparatus in which the electrodes move and cells are substantially stationary. The relative movement of cells and the electrodes is such that cells pass between the electrodes. The rate of the relative movement is more important than whether it is the cells or electrodes (or both) move.

In one embodiment, the invention involves a method for effecting electroporation that involves displacing a sample across electric field lines of a spatially inhomogeneous electric field while the field is substantially constant in terms of magnitude.

The electric field can be established by electrodes coupled to a DC source. The electric field being can be established by electrodes coupled to an AC source. The electric field can be established by electrodes having a peak power consumption not exceeding 150% of an average power consumption. The peak and average power consumption can be less than about 10 Watts. The electric field can be established by electrodes having a duty cycle greater than 50%.

In another embodiment, the invention involves a method for electroporating a sample. A spatially inhomogeneous electric field is generated with a pair of electrodes. The pair of electrodes and a sample are displaced relative to one other while the electric field is substantially constant in terms of magnitude so that the sample is displaced across electric field lines for a time sufficient to effect electroporation.

The electrode can be fixed while the sample is displaced. The sample cam be fixed while the electrode is displaced. The sample and electrode can both be displaced. The electrode can be continuously energized by a DC source of approximately 100 to 150 volts. The electrode can be continuously energized by an AC source of approximately 100 to 150 volts and a frequency of approximately 10 to 60 Hertz. The AC source can be accessed directly through a standard electrical wall outlet.

In another embodiment, the invention involves an electroporation apparatus including a channel, an inlet, an outlet, and a pair of electrodes. The channel is configured to contain a flow of particles. The inlet is in fluid communication with the channel. The outlet is in fluid communication with the channel. The pair of electrodes are adjacent the channel and generate within the flow channel a spatially inhomogeneous electric field that temporarily exposes the particles flowing through the channel to effect electroporation.

The channel can be wall-less and can include hydrophobic and hydrophilic regions. The apparatus can also include a separate cooling element in operative relation with the channel. The apparatus can also include flow shunts in operative relation with the channel.

In another embodiment, the invention involves an apparatus for electroporating a sample including a pair of electrodes and a controller. The controller is configured to displace a sample relative to one or both of the electrodes while the electrodes are continuously energized so that the sample is displaced across electric field lines for a time during which exposure to the electric field is sufficient to effect electroporation.

The controller can be a computer configured to establish a flow rate of the sample. The controller can be a computer configured to displace one or both of the electrodes.

In another embodiment, the invention involves a flow-electroporation chamber including electrodes having a peak power consumption not exceeding 150% of an average power consumption.

In another embodiment, the invention involves a flow-electroporation chamber including electrodes having a duty cycle greater than 50%.

As used herein, a “sample” means one or more cells, particles, or other materials that can be electroporated. “Displace” means the movement by any means of a sample relative to another entity, including an electric field. The term “substantially” should be given its ordinary meaning, and in preferred embodiments, a “substantially constant” quantity is a quantity that has its maximal and minimal values within 50% of its average value during a specified period of time.

Other features and associated advantages will become apparent with reference to the following detailed description of specific embodiments in connection with the accompanying drawings.

### BRIEF DESCRIPTION OF THE DRAWINGS

The following drawings form part of the present specification and are included to further demonstrate certain aspects of the present invention. Embodiments of the invention may be better understood by reference to one or more of these drawings in combination with the detailed description.

FIG. 1 is a schematic representation of a prior art flow electroporation device.

FIG. 2 is a schematic representation of a prior art flow electroporation process.

FIG. 3 shows streaming electroporation according to embodiments of the present disclosure.

FIG. 4 is an exploded perspective view of an embodiment of a streaming flow cell.

FIG. 4A is an end-view of an embodiment of a streaming flow cell.

FIG. 4B is a side-view of an embodiment of a streaming flow cell.

FIG. 5 is a histogram measured by green fluorescence on flow cytometer showing the efficiency of co-transfected cells using a flow cell and process in accordance with embodiments of the present disclosure.

FIG. 6 is a graph showing the efficiency of co-transfected cells using a flow cell and process in accordance with embodiments of the present disclosure.

FIG. 7 is a schematic of an electroporation device that uses a moving electrode tip, in accordance with embodiments of the present disclosure.

FIGS. 8 and 9 are schematics of an electroporation device that uses a wall-less design, in accordance with embodiments of the present disclosure.

FIG. 10 is a schematic of a multi-channel electroporation device, in accordance with embodiments of the present disclosure.

FIG. 11 is a schematic of an electroporation device, in accordance with embodiments of the present disclosure.

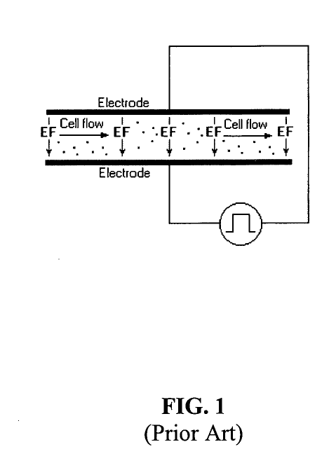

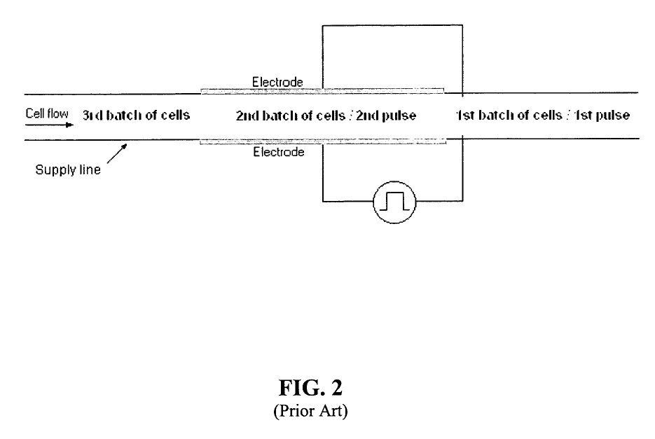

DESCRIPTION OF ILLUSTRATIVE EMBODIMENTS

Embodiments of this disclosure can be referred to as “streaming” electroporation because, in general, it is the sample streaming relative to an electric field that primarily determines the exposure of the sample to the electric field that effects electroporation. This, of course, is in contrast to conventional techniques in which the duration of an electrical pulse (or pulses) applied to electrodes primarily determines the exposure of the sample to an electric field. In other words, in streaming EP, the rate of relative motion between an electric field and a sample can be used to achieve electroporation instead of signal pulsing applied to the electrodes. As will be understood below, embodiments of this disclosure can nevertheless utilize signal pulsing, although that pulsing no longer acts as the primary mechanism for achieving electroporation.

In streaming EP, biological cells are effectively “pulsed” by their defined movement across electrical field lines (as opposed to movement with electric field lines), which in preferred but non-limiting embodiments is a substantially invariant electric field (but whose polarity may be periodically reversed). The cells pass between a pair of electrodes (e.g., very narrow electrodes), which can be connected to a DC voltage source. Other embodiments use different sources. Each cell moves across electric field lines and is exposed to an electric field for the period of time it spends between the electrodes (which is analogous to a pulse width in a typical application). The field quickly increases as the cells approach the space between the electrodes, reaches its maximum and decreases as the cells leave this space. Again, in preferred embodiments, this electric field can remain invariant.

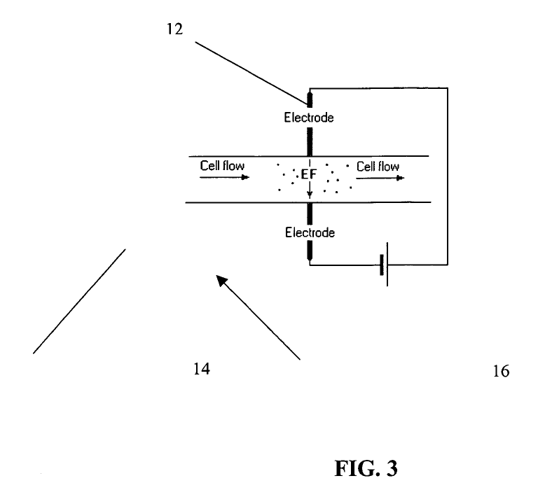

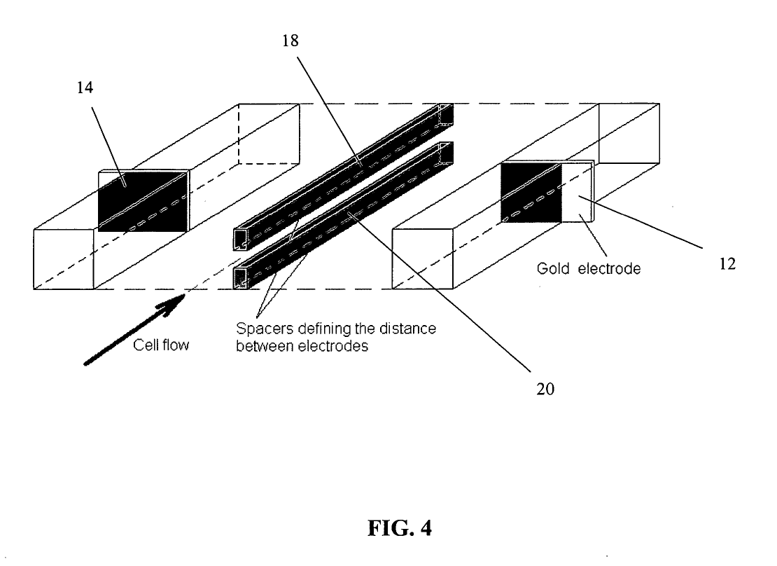

The cell exposure time equals the ratio of electrode length in the direction of flow to the linear velocity of cell movement (see FIG. 3).

Representative advantages of this streaming process are listed below:

### Duty Cycle

Streaming EP can use electrodes that are continuously energized (rather than pulsed on and off) while a sample traverses the electric field. Because cells can continuously flow between the electrodes, the electronic system never needs to be idle (since it can supply easy-to-control direct current instead of time-spaced pulses). The duty cycle of such a system is about 100 percent, as compared to 0.02 percent in a conventional flow EP application operating on the “short pulse—long wait—short pulse” principle. It will be understood by those of ordinary skill in the art having the benefit of this disclosure that electrodes can be turned off (or pulsed) occasionally and still achieve benefits of this invention and operate primarily by exposing samples based on their speed relative to electrodes. For instance, duty cycles lower than 100% yet higher than the typical 0.02% can be achieved by streaming samples and electrodes relative to one another but by periodically reducing or eliminating the energized state of the electrodes. In this way, a flow-electroporation chamber using electrodes having a duty cycle of about 90%, 80%, 70%, 60%, 50%, 40%, 30%, 20%, 10%, or 1% (and any value in between) can be achieved. In general, an electroporation chamber having a duty cycle lower than or equal to 100% but greater than or equal to 1% can be achieved using the techniques of this disclosure (e.g., exposing samples as determined by their speed relative to a passing electric field).

Even though the volume of cells between electrodes at any instant in streaming EP may be smaller than in a traditional flow embodiment, the increase in the duty cycle allows maintaining the same overall throughput or more. As the inventor's experiments indicate, the actual throughput can be substantially increased by proper choice of the flow rate and electrode/channel dimensions.

Energy Consumption

In a typical flow EP application, energy is concentrated in HV pulses. Most of this energy is dissipating in the form of heat that is produced in the cell-surrounding media. Power dissipation that slightly heats the cell suspension is an unavoidable consequence of applying an electric field, even though the electroporation itself is not caused by heating. However, the instantaneous power consumption during a pulse is huge and can be as high as 100 kilowatts. This necessitates the use of more powerful (thus heavy, bulky and expensive) electronic switching devices, energy storage components and conductors. Streaming electroporation, on the other hand, allows “spreading” this energy over a significantly larger amount of time (preferable, over the entire time of the process), thus reducing peak energy requirements in particular embodiments to about (or less than) 10 Watts (ten thousand fold less). By not having to rely on pulsed energy, the peak and average energies supplied to electrodes can be about equal. In one embodiment, the peak power consumption does not exceed 150% of an average power consumption. When pulses are used exclusively, the average energy is significantly lower than the peak energy due to the long periods of time at which the electrode is not energized at all.

Therefore there is no need to store energy and concentrate it in a high-energy pulse and use devices capable of handling big energy bursts. All necessary control over electricity can be accomplished by small inexpensive components- and the whole EP apparatus can have dimensions and weight of a cellular phone.

Throughput

Given the fact that in most EP applications it takes about 50 Joules of energy to process one milliliter of cell suspension, the theoretical limit of a preferred embodiment of streaming EP throughput is huge (e.g. limited by the DC power supply, which could easily be very large). The inventor estimates that streaming EP can process 10-50 milliliters of sample per second (up to 200 liters per hour).

One embodiment of the present invention is an electroporation device that includes walls defining a flow channel configured to receive and to transiently contain a continuous flow of a suspension comprising particles, an inlet flow portal in fluid communication with the flow channel, whereby the suspension can be introduced into the flow channel through the inlet flow portal, an outlet flow portal in fluid communication with the flow channel, whereby the suspension can be withdrawn from the flow channel through the outlet flow portal, the walls of the flow channel comprising at least a first narrow (˜0.1 mm) electrode plate on the first wall and a second narrow electrode plate on the second wall opposite the first wall; the paired electrodes are placed in electrical communication with a DC voltage source, whereby an electrical field is formed between the electrodes; whereby a suspension of cells flowing fast through the channels is continuously subjected to an electrical field formed between the electrodes, but each cell is subjected to the electric field only for the period of time that cells spend between the electrodes as it flow through the channel. In this way, while the electric field is not changing, individual cells experience the field transiently. Each cell experiences the equivalent of a pulse but no pulsing of the electrodes is required.

The conductivity of the medium in which the cells are suspended provides for a current flowing between the electrodes. Current flow through biological buffer results in a temperature increase that can damage live cells and must be limited. In a continuous process like flow EP or streaming EP, the rate of heat generation must be balanced by the rate of heat removal by cooling elements to maintain a temperature that does not damage the cells. In the simplest case, the metal electrodes themselves can serve dual purpose besides delivering an electric field to cells, they can act as heat sinks and take heat away from the buffer by virtue of the high thermal conductivity of the metal the electrodes are made of. Needing to perform each of these tasks efficiently by the same component creates serious limitations to the design of an EP channel, and optimal conditions must be found by selecting specific flow channel geometry.

In streaming EP the electrodes can be designed to be very small in relationship to flow channel dimensions, and they may not effectively remove heat. On the other hand, streaming EP offers an opportunity to approach the cooling process differently and abolish multiple design limitations. If necessary, the cell suspension can be brought in contact with any cooling element as soon as it exits the gap between electrodes (approximately 1 millisecond after being exposed to EF) or during electroporation. There is no reasonable restriction to the design of an effective heat exchanger, which can be placed downstream of the flow because it no longer has to be physically merged with the electrodes. Embodiments of the present invention therefore provide for a flow cell that is capable of removing heat more rapidly so that damage to living cells that are being electroporated may be minimized.

The current flow also can result in the production of gases, especially hydrogen and chlorine at the electrode surfaces. These gases can have a detrimental effect on the cells being electroporated and their removal as soon as possible is also desirable. As the space between the electrodes in a flow cell can be minimal in the direction of the flow, it is possible to include downstream from the electrodes flow shunts immediately along the walls of the flow channel to draw off these harmful gases. Embodiments of the present invention thereby provide an effective way to remove any byproduct gases, such as gaseous hydrogen and chlorine, from the environment of the treated cells.

In embodiments of this disclosure, electrical power can be applied to the cells essentially continuously, and cells can be electroporated at all times during the process rather than only when electrical pulses are applied to the electrodes as with current methods. Because the exposure of each particle to the electric field is primarily controlled by its movement between the electrodes, the electronic system need not be idle at any time (since electrodes can be continuously energized rather than pulsed followed by long periods of being inactive). The duty cycle of streaming EP can approximate 100% as compared to fractions of percent in current methods. Since the total power needed to electroporate a given volume of cell suspension by both methods is the same, the peak power consumption in current methods is significantly higher inversely proportional to the duty cycle than the continuous power applied in embodiments here, thereby making the present method a low-power system compared to current apparatus. This, in turn, allows for use of small, inexpensive power sources. A suitable power source could deliver 100-150 Volts DC and maintain very low current (e.g. <50 mA) during the process.

Most electroporation processes make use of exponentially decaying pulses (even if an incomplete capacitor discharge is used and a pulse has distinct leading and trailing edge). This is not because exponential pulses work best but only because it is extremely costly to build a device that generates high-power pulses of any other shape. In the most common approach, electrical energy is stored in capacitors—thus the pulse shape is exponential due to the nature of capacitive discharge. Sharp voltage transitions caused by rapid switching produce shock waves (the entire electrochemical system is being pushed away from its equilibrium), which can be dangerous to live cells. Additionally, several works have shown the benefits of using a carefully designed voltage changes realized by varying pulse number and width, as well as several pulses of different magnitude in sequence. The reason for better EP efficiency in this case originated from the optimal combination of conditions for the two essential processes: pore formation and electrophoretic transfer of charged material, such as DNA, to the cell surface and through the lipid membrane. A difficulty associated with adjustment of the voltage time course is related to having to use several power supplies (capacitor-switch pairs) in accordance with the number of pulses.

In the case of streaming EP cells are virtually “pulsed” by their movement through an electrical field. The field quickly increases as the cells approach the electrodes, reaches its maximum and decreases as the cells are moved away from the electric field. The time course of field intensity across each cell can be described approximately by a bell-shaped curve. Its half-width will depend on the rate of passage of the cells between the electrodes and electrode spacing and dimensions. Changing the shape of the electrodes will change the shape of the electric field (producing faster raise/slower decay or vice versa). In an apparatus in which cells flow between the electrodes through a channel, positioning multiple electrode pairs in sequence in the flow can result in multiple pulses being applied to each cell.

While application of a time invariant electric field allows embodiments to be operative, such operation can result in polarization of the electrodes. It is well known in electrochemistry that while electrode polarization cannot be entirely prevented, it can be minimized by periodic reversal of the electrode potential. Alternating the polarity should occur on a longer time scale than the duration of each cell's transit through the field so that the exposure of cells to the electric field remains controlled primarily by their relative movement rather than by electronic waveform generators. For example, if it takes 1 millisecond for each cell to pass between electrodes, and one reverses the polarity of voltage every 100 milliseconds, then about 100 elementary volumes of cell suspension will be processed during the time between subsequent changes of voltage polarity. But this rate of polarity reversal (10 times a second) must be fully sufficient to prevent significant electrode polarization. Because of the low power consumption of the process, the voltage polarity reversal can be easily done by an inexpensive semiconductor device.

In one embodiment, a convenient way to obtain a reversing electrical field is to connect to an ordinary power line alternating current (AC) that is widely available at 110 or 220 V (RMS). This current varies with a frequency of 60 to 50 cycles per second (depending on the utility). The duration of time during which the voltage is higher than EP threshold is long (on the order of 20 milliseconds) compared to the transit time for a cell passing between the electrodes (<1 millisecond), thus the exposure of each particle or cell to the electric field is controlled by its movement between the electrodes. If the spacing between the electrodes is on the order of one millimeter the voltage supplied by the utility can be used directly to provide an electric field of 1000 to 2000 volts per centimeter, which is within the range most useful for electroporation of most cell types. In the above configuration an electric power supply, at least an electric power supply owned by the user of the apparatus, is essentially eliminated. If necessary, the electroporation apparatus can be directly connected to the power line and remain functional. Even though in this embodiment not every cell passing between the electrodes will necessarily experience the same electric field in terms of duration and field strength, a high percentage of cells will experience an electric field having a duration and intensity needed to effect electroporation.

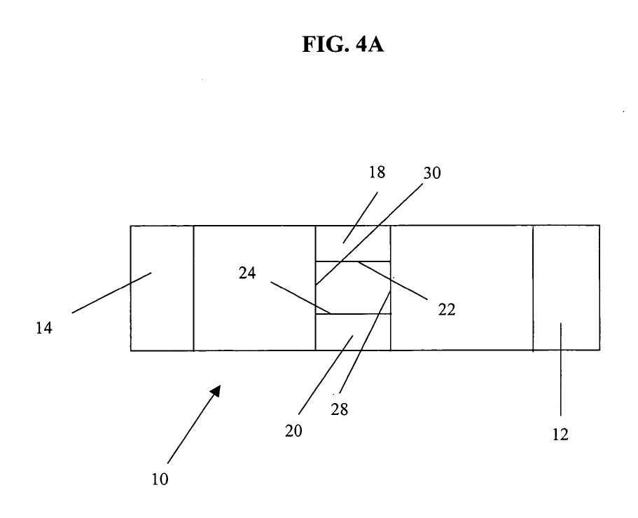

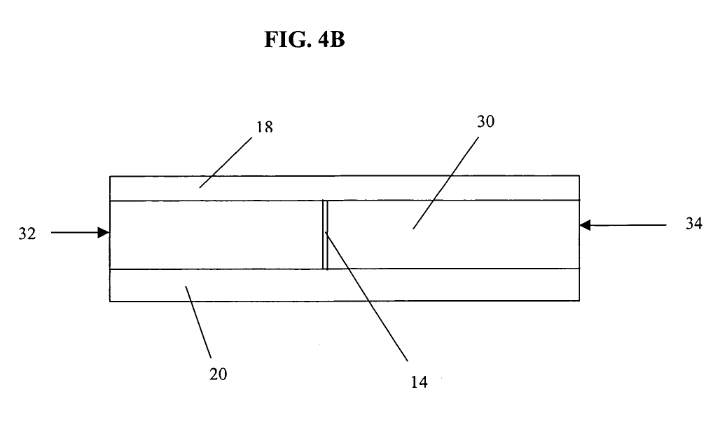

With reference to FIGS. 3 and 4 in which like numbers indicate like elements throughout the several views, there is disclosed a streaming electroporation cell assembly 10 that includes two opposing electrodes 12, 14. Typically, the electrodes 12, 14 may be constructed of gold, platinum, carbon or other electrically conductive insoluble materials.

In alternate embodiments, one or both of the opposing electrodes 12, 14 may further be positioned next to one or more cooling elements (see cooling element 17 of FIG. 3). The cooling element may be a thermoelectric cooling element, or may provide cooling by direct water or other coolant contact, by ventilation through a heat sink, or other cooling means to dissipate heat generated in the electroporation process.

Referring to FIGS. 4, 4A and 4B, the electrodes 12, 14 may typically be separated by one or more electrode gap spacers 18, 20. The thickness of the electrode gap spacers 18, 20 will define and fix a gap 22 between the electrodes 12, 14. The gap 22 between the electrodes 12, 14 can easily be adjusted to any desired measurement simply by changing the gap spacers 18, 20. The thickness of one such gap 22 will vary depending on the flow rate and voltage to be applied between the electrodes.

Each of the electrode gap spacers 18, 20 defines a wall 22, 24. There is a central sample well and insulating side walls 28, 30. The electrode gap spacers also define a fluid inlet 32. The fluid inlets 32 permit fluid to be introduced to the central sample well and to contact the walls 22, 24, 26, 28. The electrode gap spacers 18, 20 also define a fluid outlet 34. The fluid outlet 34 permits fluid in the central well to be removed or to exit therefrom. The electrode gap spacers 18, 20 are typically constructed of an electrically insulating material, and may be fashioned from such materials as plastic, ceramic, rubber, or other non-conductive polymeric materials or other materials.

In various embodiments of the flow electroporation cell assembly, each flow electroporation cell assembly may contain a single flow channel or a plurality of flow channels oriented between the opposing electrode plates. When desirable, multiple flow channels may be provided to achieve more rapid, higher volume electroporation treatment. It may be desirable that at least two opposed electrodes are embedded in a portion of the opposed walls of the electroporation region of the flow channel. The term “electroporation region” as used herein means that portion of the flow channel in which material flowing therethrough is exposed to an electric field of sufficient strength to effect electroporation. It is not necessary that either or both of the electrodes be embedded in the opposed walls. Further a flow channel includes any space between electrodes, and such flow channel need not be defined by physical walls.

Preferably, a flow electroporation cell assembly may be provided as a sterile unit for disposable, single-use applications. The components of the flow electroporation cell assembly may thus preferably be constructed of materials capable of withstanding sterilization procedures, such as autoclaving, irradiation, or chemical sterilization.

While one application of this invention is to effect the electroporetic transfer of materials, particularly DNA, into cells, it is recognized that application of an electric field to living cells can have other effects. Included among those effects is killing of the cells. While in the case of electroporetic transfer of DNA into cells killing of cells is undesirable, under other circumstances killing of cells may be the desired outcome. Application of electric fields of higher intensity and duration than is optimal for electroporation does result in cell killing and such intensities and durations can be provided using techniques of the present invention. Sterilization of materials to effect killing of infectious cells can therefore be carried out using the present invention. Further, the optimal duration and magnitude of the electrical field may vary according to the type of cell being treated and the result desired as a consequence of the treatment. The present invention is not limited in any way by the duration or magnitude of the electric field, and the method is intended to apply to any cell or cell-like particle being treated. In fact, the present in invention can find utility in any process where transient application of an electric field to a particle is desired.

With the benefit of the present disclosure, those of ordinary skill in the art will comprehend that techniques claimed here and described above may be modified and applied to a number of additional, different applications, achieving the same or a similar result. The claims attached hereto cover all such modifications that fall within the scope and spirit of this disclosure.

The following examples are included to demonstrate specific, but non-limiting embodiments of this disclosure. It should be appreciated by those of skill in the art that the techniques disclosed in the examples that follow represent techniques discovered by the inventor to function well in the practice of the invention, and thus can be considered to constitute specific modes for its practice. However, those of ordinary skill in the art should, in light of the present disclosure, appreciate that many changes can be made in the specific embodiments which are disclosed and still obtain a like or similar result without departing from the spirit and scope of the invention.

### EXAMPLE 1

A flow cell was built as illustrated in FIG. 4. The flow cell was built with the following dimensions:

| | | |
|---|---|---|
| Electrode width: | 0.1 | um (the dimension in the |
| | | direction of flow) |
| Electrode material: | 99.985% | gold |
| Distance between electrodes: | 1 | mm |
| Channel height: | 3 | mm |

The flow cell was tested in a process of transfecting Jurkat cells (˜5×106/mL) with a GFP-encoding plasmid (100 ug/mL) under the following conditions:

| | | |
|---|---|---|
| Flow rate: | 11.5 | ml/min |
| Voltage applied to electrodes: | 100 | V DC |
| Volume of sample: | 1.5 | ml |

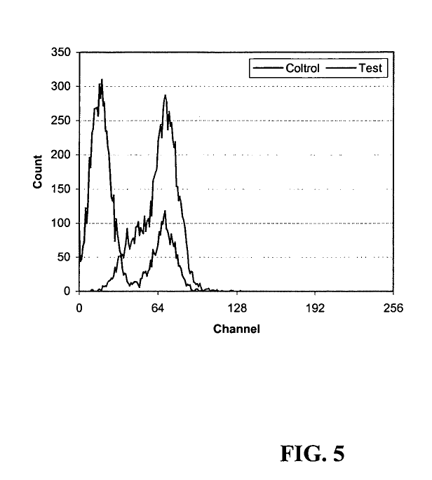

As illustrated in FIG. 5, after 24 hours, about 95% of the Jurkat cells were GFP-positive demonstrating electroporetic cell transformation.

### EXAMPLE 2

A flow cell was built with the following dimensions:

| | | |
|---|---|---|
| Electrode width: | 0.1 | μm (the dimension in the |
| | | direction of flow) |
| Electrode material: | 99.985% | gold |
| Distance between electrodes: | 1 | mm |
| Channel height: | 3 | mm |

It was tested in a process of transfecting Jurkat cells (˜5×106/mL) with GFP-encoding plasmid (100 μg/mL) under the following conditions:

| | | |
|---|---|---|
| Flow rate: | 12 | ml/min |
| Voltage applied to electrodes: | 110 | V AC |
| Volume of sample: | 2 | ml |
| Flow rate: | 20 | ml/min |
| Voltage applied to electrodes: | 110 | V AC |
| Volume of sample: | 2 | ml |

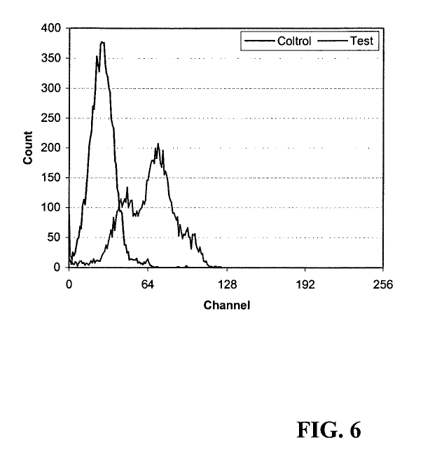

As illustrated in FIG. 6, using a residential or industrial power line as a 110 V (RMS) voltage source, after 24 hours, about 70% of the Jurkat cells at a flow rate of 12 mmin were GFP-positive. It was slightly lower at 20 mmin flow rate; these conditions corresponded to a “shorter” pulse.

In the second example above, the electrodes were directly connected to the AC electrical power supplied to the laboratory; the only additional components being a switch (for safety and convenience) and a 4 uF capacitor connected in series with the flow cell (used as a ballast to effect a voltage drop of about 50 V since the peak voltage in the power line is 150-160 Volts instead of 100 V). This capacitor would be unnecessary if the spacing between the electrodes were increased from 1 mm to 1.5 mm. It would thereby be possible to connect the electrodes directly to a wall socket (outlet receptacle) with only an ordinary switch interposed to turn the apparatus on and off.

The foregoing examples disclose particular electrode configurations, means to cause cells to pass through an electric field and electronic circuitry to produce a suitable electric field.

### EXAMPLE 3

### Moving Tip

In the following embodiment, cells to be electroporated are located near and may be attached to a conductive surface at the bottom of a culture dish or other surface. The conductive surface could actually be the bottom of the culture dish. In this embodiment the cells can remain essentially immobile during electroporation. Electroporation is accomplished by moving an electric field over the cells to provide a transient electric field to each cell.

The conductive surface below the cells in this embodiment (e.g., the bottom of the culture dish or a sheet of highly conductive material placed on the bottom of the dish), serves as an electrode. Alternatively, an actual electrode can be used. A second electrode is placed into or near the culture dish so that the tip of the electrode is near the bottom of the plate or the insert. The cells in the dish can be submerged in growth media or a medium formulated to maximize electroporation to a depth such that both the cells and the tip of the pin-like electrode are submerged. The part of the tip that is likely to be submerged can be coated with gold or a similar metal on all surfaces that are conductive with the medium.

To effect electroporation, a voltage is applied between the pin-like electrode and the conductive surface below the cells, and the tip is moved. Preferably, it is moved to maintain a predetermined and constant distance with the conductive surface. The rate of movement of the tip can be adjusted so that the duration of the electric field experienced by any cell located below the tip as it travels is optimal for electroporation. The distance between the end of the tip and the conductive surface below the cells (or the other electrode) is chosen to provide an electric field to the cells that has a magnitude sufficient for electroporation.

The path of the moving electrode can be chosen so that the tip passes over every cell once before it passes over any cell twice assuming that such a second pass is desired. This path can be substantially horizontal as the plate bottom and the conductive surface can be horizontal to provide a uniform fluid depth over the conductive surface. The path taken by the pin-like electrode can be raster-like if a square shaped culture dish is used or spiral if a round culture dish is used.

As the electrode travels, a current can be measured to reflect any changes in the distance between the electrodes. Small and gradual changes in the current would presumably be caused by changes in the distance between the electrodes resulting from non-flatness of the conductive surface or the surface not being level, and the pin-like electrode could be raised or lowered in its path to maintain a constant distance between the electrodes. This correction could be controlled electronically using a computer and appropriate programs. Minor adjustment to the height of the pin-like electrode could be accomplished using one or more piezoelectric devices or other steppers.

As with other embodiments, periodic reversal of the polarity of the electrodes can be employed to minimize electrode polarization. Similarly, alternating current (e.g., as provided by a utility) could be used for convenience. When using alternating current it would be possible, and may be desirable, to have the movement of the pin-like electrode halt briefly whenever the polarity is switching. By doing this, one may avoid having the pin-like electrode pass over a cell when the electric field between the electrodes is too weak or is reversing and is therefore incapable of electroporating such a cell. This embodiment may be employed using more than one pin-like electrode per plate.

The cells may not need to be located immediately atop the conductive surface and for some applications it may be desirable to have a matrix of protein or carbohydrate between the cells and the conductive surface.

In one embodiment, only one of the electrodes is pin or wire like, and the other can include a plate having a surface far larger than the surface of the other electrode. The electric field resulting when these electrodes are brought together will have a different shape than that produced between two pin-like or wire-like electrodes.

The distance the pin-like electrode travels to provide an electric field of the desired magnitude will depend at least on the optimal electric field strength for the sample being electroporated, the conductivity of the media, the distance between the pin-like electrode's path and the conductive surface, and the distance between the pin-like electrode's path and the cells. This method can be particularly effective when the cells comprise a monolayer or have not yet grown sufficiently to quite achieve a monolayer.

Any non-pin-like electrode need not be flat. It can have any shape. Preferably, it allows cells to be located close to it and at a uniform distance from it. Use of a non-flat surface is likely to make the process of keeping media over the cells and moving the pin-like electrode more complicated and difficult. But, use of a non-flat surface is possible. It fact, most surfaces that can practically be employed will not be absolutely flat. As discussed above, even non-flatness can readily be compensated for.

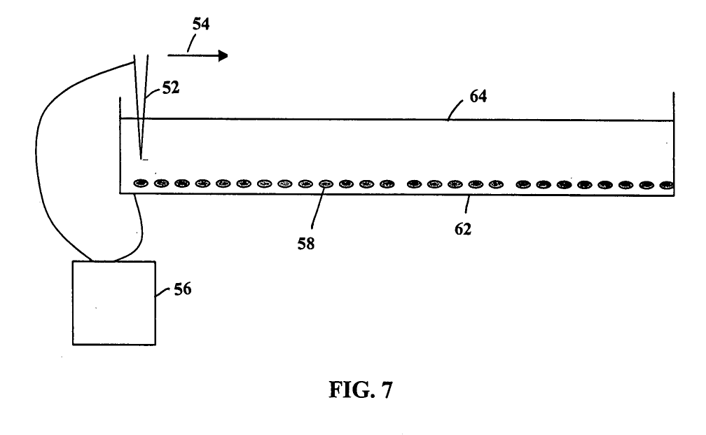

FIG. 7 illustrates an embodiment involving a moving electrode with a fixed sample. Mobile electrode 52 moves in the direction of arrow 54. The electrode is energized by voltage source 56, which may be DC or AC. Dish bottom 62 serves as another electrode. Cells 58 are temporarily exposed to an electric field as the mobile electrode 52 passes over them. The exposure can be varied by varying the speed of movement. The speed is chosen to effect electroporation. A media surface 64 is shown above the cells 58.

### EXAMPLE 4

### Wall-Less Flow EP

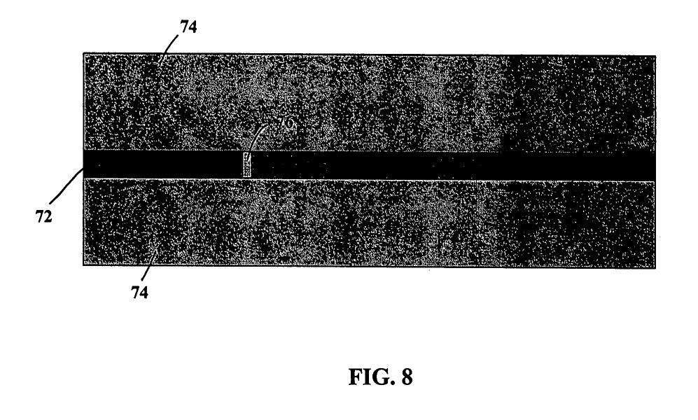

FIG. 8 shows an embodiment in which a channel does not utilize traditional walls. In FIG. 8, hydrophilie channel 72 is surrounded by hydrophobic regions 74. An electrode 76 is shown in operative relation with the hydrophilic channel 72.

To implement the embodiment illustrated in FIG. 8, one may match two mating plates having a hydrophobic length-wise channel to create a space in which a solution is constrained by surface tension. A pair or more of electrodes can be located opposite one another about the hydrophilic channel. In wall-less embodiments such as these, there need not be any traditional, physical walls.

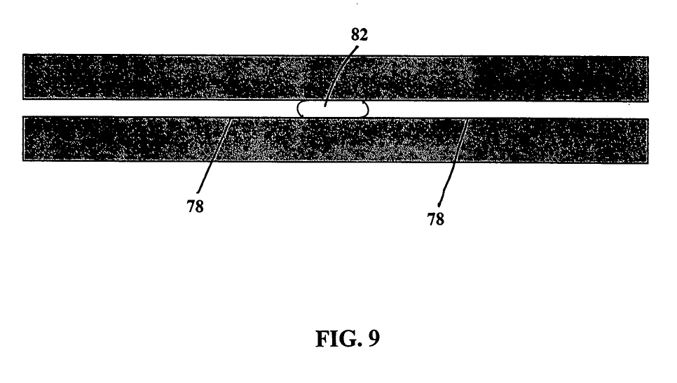

FIG. 9 shows an end view of a suitable embodiment, in which hydrophobic surfaces 78 and a hydrophilic channel constrain fluid 82 to flow only along the hydrophilic channel (in this figure, fluid 82 is flowing into or out of the page).

### EXAMPLE 5

### Parallel Multi-Channel Streaming

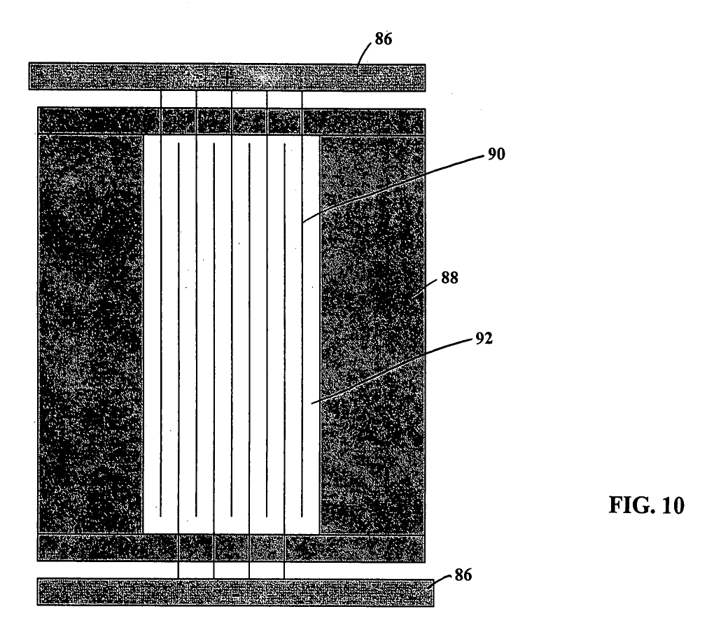

FIG. 10 shows an embodiment in several channels are used for streaming EP. Shown are source 86, electrode wires 90, non-conducting material 88, and channels 92 (each space between wires 90 represents a channel).

In this embodiment, all cells can flow down a single, master channel comprised of all the individual channels. Adjacent wire electrodes have opposite polarities. Overall polarities can be switched to avoid polarization. Bulk flow can be very high with moderate linear velocity and reduced wall effects using this multi-channel concept.

### EXAMPLE 6

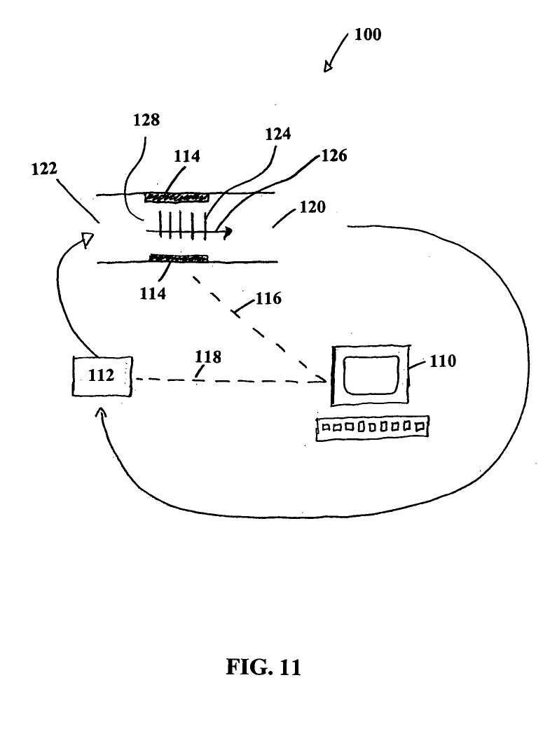

FIG. 11 shows a general embodiment illustrating several aspects of embodiments of this disclosure. Shown is a system 100 including electrodes 114, inlet 122, outlet 120, pump 112, channel 128, and controller 110 that communicates with electrodes 114 via link 116 and with pump 112 via link 118.

Controller 110 can be a computer, controller card, or any other device suitable for influencing pump 112 to establish a flow rate (an example flow show by the arrows coming from and to pump 112) suitable for streaming EP and/or for displacing electrodes at a rate suitable for streaming EP. In particular, controller 110 can control pump 112 to establish a flow rate such that a sample flowing between electrodes 114 is only exposed to the associated electric field for a time sufficient to effect electroporation. Alternatively, controller 110 can displace one or both of electrodes 114 relative to the sample so that their electric field passes over the sample for only a time sufficient to effect electroporation. Alternatively, controller 110 can control both pump 112 and electrodes 114 together to ensure a suitable relative rate of movement is established for streaming EP.

Links 116 and s118 can be hard-wired, wireless, or any other type known in the art. Controller 110 can run appropriate software, firmware, or built-in algorithms to facilitate its control.

FIG. 11 shows example electric field lines 124. It will be understood by those of ordinary skill in the art that these are just examples and that significantly different electric field distributions may be set up to effect electroporation. Suitable commercial programs can be used to model the electric field within a channel and to arrive at actual electric field lines that accurately reflect the physical geometries and electrical parameters of a particular channel. Arrows 126 in FIG. 11 demonstrate how a sample can travel across the electric field lines 124, as opposed to traveling substantially with those field lines. The transversal need not be perpendicular, although that is how it is illustrated in FIG. 11 for convenience. The transversal can be effected by having the sample flow through the channel or having the electrode(s) move relative to the sample, or both. Electric field lines 124 can represent a spatially inhomogeneous or invariant field. Electric fields in a region between the electrodes 114 can be substantially constant in terms of magnitude.

Electrodes 114 can be coupled to a DC source or an. AC source to establish the electric field. As discussed before, electrodes 114 can have a peak and average power consumption that are about equal, and in a preferred embodiment, this consumption is less than about 10 Watts. The duty cycle of electrodes 114 can be about 100% and in preferred embodiments greater than 50%.

In one embodiment, electrodes 114 are continuously energized. In other words, electrodes 114 remain on at least for the time period in which the sample is moving through the electric field. In this embodiment, electrodes 114 are not energized to create a pulse, then turned off to wait a certain amount of time, and then energized again to create another pulse. Instead, they are continuously energized by, for example, being connected to a DC or AC source. In this way, the total and average energy consumption can be “evened out,” as discussed above. In this way also, the duty cycle can be significantly higher than in conventional systems.

With the benefit of the present disclosure, those having skill in the art will comprehend that techniques claimed herein may be modified and applied to a number of additional, different applications, achieving the same or a similar result. The claims attached hereto cover all such modifications that fall within the scope and spirit of this disclosure.

### REFERENCES

Each of the following references is hereby incorporated by reference in its entirety:

U.S. Pat. No. 4,220,916

U.S. Pat. No. 6,077,479

U.S. Pat. No. 6,617,154

U.S. Pat. No. 6,485,961

U.S. Pat. No. 6,074,605

U.S. Pat. No. 5,720,921

1. A method comprising effecting electroporation by displacing a sample across electric field lines of a spatially inhomogeneous electric field while the field is substantially constant in terms of magnitude.

2. The method of claim 1, the electric field being established by electrodes coupled to a DC source.

3. The method of claim 1, the electric field being established by electrodes coupled to an AC source.

4. The method of claim 1, the electric field being established by electrodes having a peak power consumption not exceeding 150% of an average power consumption.

5. The method of claim 4, where the peak and average power consumption are less than about 10 Watts.

6. The method of claim 1, the electric field being established by electrodes having a duty cycle greater than 50%.

7. A method for electroporating a sample, the method comprising:
generating a spatially inhomogeneous electric field with a pair of electrodes; and
displacing the pair of electrodes and a sample relative to one other while the electric field is substantially constant in terms of magnitude so that the sample is displaced across electric field lines for a time sufficient to effect electroporation.

8. The method of claim 7, where the electrode is fixed and the sample is displaced.

9. The method of claim 7, where the sample is fixed and the electrode is displaced.

10. The method of claim 7, where the sample and electrode are both displaced.

11. The method of claim 7, where the electrode is continuously energized by a DC source of approximately 100 to 150 volts.

12. The method of claim 7, where the electrode is continuously energized by an AC source of approximately 100 to 150 volts and a frequency of approximately 10 to 60 Hertz.

13. The method of claim 12, where the AC source is accessed directly through a standard electrical wall outlet.

14. The method of claim 7, the electrode having a peak power consumption not exceeding 150% of an average power consumption.

15. The method of claim 14, where the peak and average power consumption are less than about 10 Watts.

16. The method of claim 7, the electrode having a duty cycle greater than 50%.

17. An electroporation apparatus comprising:
a channel configured to contain a flow of particles;
an inlet in fluid communication with the channel;
an outlet in fluid communication with the channel; and
a pair of electrodes adjacent the channel that generate within the flow channel a spatially inhomogeneous electric field that temporarily exposes the particles flowing through the channel to effect electroporation.

18. The apparatus of claim 17, the channel being wall-less and comprising hydrophobic and hydrophilic regions.

19. The apparatus of claim 17, the electrodes having a peak power consumption not exceeding 150% of an average power consumption.

20. The apparatus of claim 19, where the peak and average power consumption are less than about 10 Watts.

21. The apparatus of claim 17, where the electrodes have a duty cycle greater than 50%.

22. The apparatus of claim 17, further comprising a separate cooling element in operative relation with the channel.

23. The apparatus of claim 17, further comprising flow shunts in operative relation with the channel.

24. An apparatus for electroporating a sample, the apparatus comprising:
a pair of electrodes; and
a controller configured to displace a sample relative to one or both of the electrodes while the electrodes are continuously energized so that the sample is displaced across electric field lines for a time during which exposure to the electric field is sufficient to effect electroporation.

25. The apparatus of claim 24, where the controller comprises a computer configured to establish a flow rate of the sample.

26. The apparatus of claim 24, where the controller comprises a computer configured to displace one or both of the electrodes.

27. The apparatus of claim 24, the electrodes having a peak power consumption not exceeding 150% of an average power consumption.

28. The apparatus of claim 27, where the peak and average power consumption are less than about 10 Watts.

29. The apparatus of claim 24, where the electrodes have a duty cycle greater than 50%.

30. The apparatus of claim 24, further comprising a separate cooling element configured to cool the sample during or following electroporation.

31. A flow-electroporation chamber comprising electrodes having a peak power consumption not exceeding 150% of an average power consumption.

32. The flow-electroporation chamber of claim 31, where the peak and average power consumption are less than about 10 Watts.

33. A flow-electroporation chamber comprising electrodes having a duty cycle greater than 50%.
